(19) United States
(12) Patent Application Publication
(10) Pub. No.: US 2006/0217720 A1
(43) Pub. Date: Sep. 28, 2006

(54) Artificial hip joint without a shaft

(76) Inventor: Poonung Chieng, Shanghai (CN)

Correspondence Address: BACON & THOMAS, PLLC, 625 SLATERS LANE, FOURTH FLOOR, ALEXANDRIA, VA, 22314, US

(21) Appl. No.: 10/549,255

(22) PCT Filed: Mar. 15, 2004
(86) PCT No.: PCT/CN04/00206

§ 371 Date: Sep. 13, 2005

(30) Foreign Application Priority Data
Mar. 13, 2003 (CN) 03115792.0

(51) Int. Cl. A61F 2/30

(52) U.S. Cl. 606/65

(57) ABSTRACT

The invention provides an artificial hip joint without a shaft which includes an enhancing bone plate for supporting the femur, the plate having more than one hole on its surface, fixing elements for fixing the plate on the surface of the femur extending through these holes respectively; an enhancing bone screw which is expended through the plate and the femur; a center bone screw which is screwed with the enhancing bone screw; the other end of the center bone screw extending out of the femur neck; and a femur neck protecting device for protecting the femur neck, the device being fixed on the end which is not connected with the enhancing bone screw. With the fixation of the center bone screw, the protecting device can ride against the surface of the femur tightly so that it can protect the remaining bone cutting off the diseased or damaged. It can also enhance the rigidity and stabilization of the bone which is not well in function by the support of the enhancing bone plate.

### BACKGROUND OF THE INVENTION

1. Field of the Invention

The present invention relates to a device for connecting a damaged or broken femur head, more particularly to an artificial hip joint without a shaft.

2. Description of the Related Art

For the traditional treatment of a fractured or broken femur neck, the damaged or broken part is generally resumed to its original position and covered by a sheet steel, and then two nails are installed into the femur for fixing the sheet steel. However, the design of such a traditional structure has the shortcomings of being unable to stably cover the sheet steel and the newly grown blood vessels and bone scars cannot cover the sheet steel effectively, so that the sheet steel cannot be integrated completely with the femur in order to protect the femur. The force exerted on the sheet steel at the broken femur is very limited, and thus the sheet steel will be separated from the femur easily. Further, the application becomes impractical, and patients suffer from pains. The abovementioned shortcomings have been major issues for medical people and patients for years.

Another femur neck protecting device comes with a simple hollow body to protect the femur neck, and the hollow body is fixed onto the femur neck by a central screw rod. Such arrangement may be very effective for young patients, but the central screw rod may be loosened in some occasions, particularly for older patients with osteoporosis. Furthermore, the femur neck protecting device does not come with a device to protect the femur, and thus will slow down the recovery of a patient, particularly for patients having osteoporosis.

### SUMMARY OF THE INVENTION

Therefore, it is the primary objective of the present invention to overcome the foregoing shortcomings by providing an artificial hip joint without a shaft that can improve the fixing of the femur neck and enhance the strength of the femur neck, and the artificial hip joint without a shaft can be integrated with the femur of a patient.

To solve the foregoing technical problem, the present invention provides an artificial hip joint without a shaft that installs an enhancing bone plate for passing through an enhancing bone screw, and the enhancing bone screw is connected to the center bone screw and the center bone screw is connected to a femur neck protecting device. When the artificial hip joint without a shaft is in use, the femur neck protecting device is sheathed onto the cut or damaged part of the femur while the enhancing bone plate is fixed onto the femur and then the enhancing bone screw is penetrated deeply into the femur and secured with the center bone screw, and the other end of the center bone screw is passed through the femur neck protecting device.

With the fixing of the center bone screw, the femur neck protecting device can be attached closely onto the surface of the femur to protect the cut or damaged femur head and improve the rigidity and stabilization of a weak femur head under the protection of the enhancing bone plate.

The present invention also provides an artificial hip joint without a shaft that comprises a hollow sleeve disposed on a femur neck protecting device, and the hollow sleeve includes an externally extended and slightly curved flange disposed at an end of the periphery of the hollow sleeve and a neck section protruded from another end of the hollow sleeve, and the neck section has a bolt passing through the sleeve, and the sleeve and flange include at least one hole. When the artificial hip joint without a shaft is in use, the injured or damaged femur head or femur neck is cut by a surgery knife into a shape corresponding to the shape of the sleeve, such that the sleeve can be sheathed onto the cut femur neck, and the flange of the sleeve can be attached closely onto the surface of the femur, and the sleeve is secured onto the femur neck connected to the injured femur head by the bolt at the top.

Such arrangement can protect the femur neck from being hurt again, and the newly grown blood vessels and bone scars can pass through the hole and cover onto a main body so as to integrally integrate the main body with the femur.

### BRIEF DESCRIPTION OF THE DRAWINGS

FIG. 1 is an exploded view of the present invention;

FIG. 2 is a perspective view of the present invention; and

FIG. 3 is a cross-sectional view of assembled parts of the present invention.

### DETAILED DESCRIPTION OF THE PREFERRED EMBODIMENTS

The above and other objects, features and advantages of the present invention will become apparent from the following detailed description taken with the accompanying drawing.

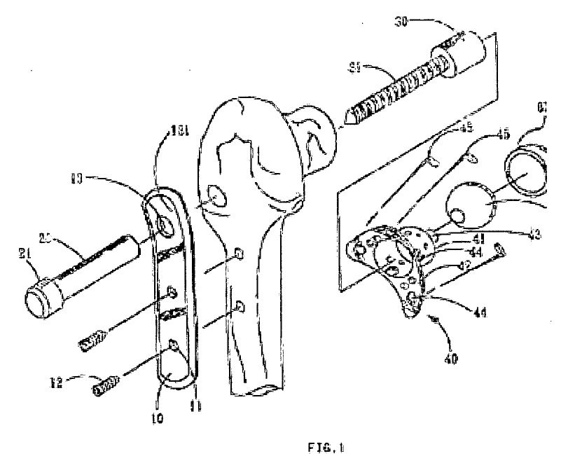

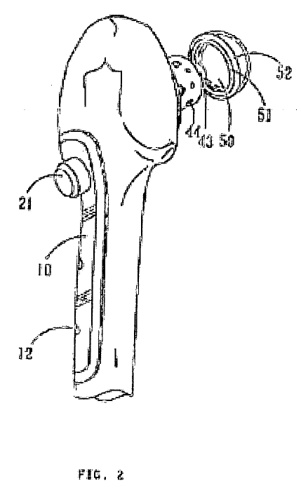

Referring to FIGS. 1 to 3 for the artificial hip joint without a shaft of the present invention, the device comprises an enhancing bone plate 10, and the enhancing bone plate 10 includes more than one hole 111, and the holes 111 separately include a fixing element 12 which is a screw in this preferred embodiment. The enhancing bone plate 10 includes an opening hole 13 disposed at a predetermined position, and an enhancing bone screw 20 passes through the opening hole 13, and the enhancing bone screw 20 is a sleeve in this preferred embodiment. The enhancing bone screw 20 has a head section 21 disposed at an end having an area larger than the area of the opening hole 13, so that when the enhancing bone screw 20 is passed into the opening hole 13, the enhancing bone screw 20 will not be sunk into the opening hole 13. The enhancing bone screw 20 further includes an internal thread for being screwed with a center bone screw 30. The center bone screw 30 has an external thread disposed at its periphery for connecting another end of the center bone screw 30 with a femur neck protecting device 40. When the artificial hip joint without a shaft is in use, the enhancing bone plate 10 is attached onto the femur first, and then the fixing element 12 is passed through the hole 11 of the enhancing bone plate 10 and fixed onto the femur. The enhancing bone screw 20 is passed through the opening hole 13 and penetrated into the femur to couple with a center bone screw 30. Another end of the center bone screw is sheathed onto the femur neck protecting device 40. With the fixing of the center bone screw 30, the femur neck protecting device can be attached closely onto the surface of the femur to fully protect the cut femur head or the damaged femur neck. By the support of the enhancing bone plate 10 for a weak femur, the rigidity and stabilization of the femur can be improved. As a result, the femur neck protecting device will not be pressed or will not be loosened by pressing, when the patient is exercising or carrying a heavy load.

Further, the femur neck protecting device 40 is a means capable of securely covering the femur neck, and the newly grown blood vessels or bone scars can be integrated. The femur neck protecting device 40 further comprises a hollow sleeve 41, and an outwardly extended and slightly curved flange 42 disposed on the periphery at an end of the sleeve 41. The sleeve 41 is in a triangular shape in this preferred embodiment. Another end of the sleeve 41 includes an inwardly withdrawn neck section 43, and the neck section 43 is engaged precisely with the center bone screw 30. The sleeve 41 and the flange 42 further include at least one hole 44 for passing a fixing element 45. The fixing element 45 is a steel nail in this preferred embodiment. However, the persons skilled in the art can substitute the steel nail with other equivalent devices.

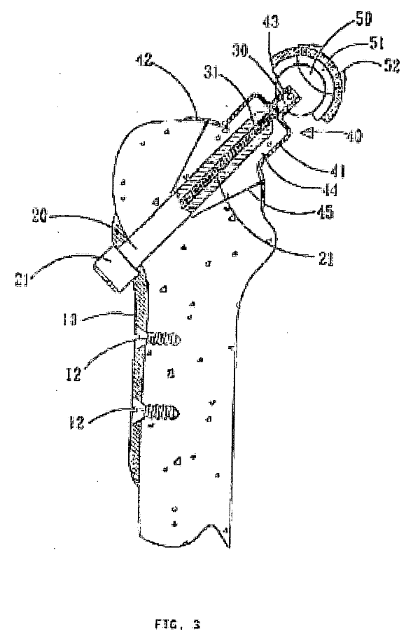

Referring to FIG. 3 for the use of the present invention, the damaged femur head or femur neck is cut into the shape of the sleeve 41 by a surgery knife first, and then the sleeve 41 is sheathed onto the cut femur head, and the flange 42 is attached closely onto the surface where the femur head and the femur are connected. Such arrangement not only secures the femur neck protecting device onto the broken femur neck, but also can protect the femur neck from being injured again by the sleeve 41. With the fixing element 45 passed through the hole 44 and fixed onto the femur, the connection between the femur and the femoral joint (not shown in the figure) can be enhanced.

Further the hole 44 of the femur neck protecting device 40 allows the newly grown blood vessels and bone scars to pass through and cover onto the flange 42 and the sleeve 41, so as to integrate with the femur neck and the femur. Further, the neck section 43 includes an adjustable joint 50 disposed on an end away from the neck section 43 for coupling with the center bone screw 30. The adjustable joint 50 includes a hip joint cup 60 covered onto the adjustable joint 50, and the hip joint cup 60 includes a plastic lining 61 which is sheathed onto a metal hip joint cup 62, so as to movably connected with the hip joint.

What is claimed is:

1. An artificial hip joint without a shaft, comprising:
an enhancing bone plate, for supporting a femur, and said enhancing bone plate including more than one hole for passing a fixing element capable of fixing said enhancing bone plate onto the surface of said femur;
an enhancing bone screw, for passing through said enhancing bone plate and into said femur;
a center bone screw, being screwed with said enhancing bone screw, and another end of said center bone screw being exposed from a femur neck; and
a femur neck protecting device, being coupled to another end that said center bone screw is not coupled with said enhancing bone screw, for protecting said femur neck.

2. The artificial hip joint without a shaft of claim 1, wherein said fixing element is a screw.

3. The artificial hip joint without a shaft of claim 1, wherein said femur neck protecting device comprises a hollow sleeve, and said hollow sleeve includes an externally extended and slightly curved flange disposed at an end of the periphery of said hollow sleeve and an inwardly withdrawn neck section protruded from another end of said hollow sleeve, such that said neck section is coupled precisely to said center bone screw, and said sleeve includes at least one hole disposed at said flange.
